(19) United States
(12) Patent Application Publication
(10) Pub. No.: US 2022/0003735 A1
(43) Pub. Date: Jan. 6, 2022

(54) ELECTROCHEMICAL MICROBIAL SENSOR

(71) Applicant: Ohio University, Athens, OH (US)

(72) Inventor: Gerardine G. Botte, Lubbock, TX (US)

(21) Appl. No.: 17/281,278

(22) PCT Filed: Oct. 1, 2019
(86) PCT No.: PCT/US2019/053997

§ 371 Date: Mar. 30, 2021

Related U.S. Application Data
(60) Provisional application No. 62/739,430, filed on Oct. 1, 2018.

(51) Int. Cl. G01N 33/02; G01N 27/416; G01N 27/407; G01N 27/327; G01N 27/30

(52) U.S. Cl. CPC G01N 33/025; G01N 27/4161; G01N 27/308; G01N 27/327; G01N 27/4076

(57) ABSTRACT

An electrochemical sensor, including a working electrode, a reference electrode, and a counter electrode. The working electrode may include a transition metal, and is contacted with a solution including an alkaline media for oxidation of the transition metal, such that the sensor may be used to provide data to quantify the amount of a pathogen in the solution. In certain embodiments, the transition metal of the working electrode is nickel. In other embodiments, the working electrode includes graphene-layered nickel. And, in certain embodiments, the working electrode may be a rotating disk electrode, wherein the working electrode rotates in a solution including an alkaline media.

### CROSS-REFERENCE TO RELATED APPLICATIONS

The present application claims priority to, and benefit of the filing date of, U.S. Provisional Patent Application Ser. No. 62/739,430, filed Oct. 1, 2018, the disclosure of which is hereby incorporated herein by reference in its entirety.

### FIELD OF THE INVENTION

Various aspects of the present invention are generally directed to electrochemical biosensors, and more specifically to electrochemical biosensors for the detection of pathogens in food and other areas.

### BACKGROUND OF THE INVENTION

This section is intended to introduce the reader to various aspects of art that may be related to various aspects of the present invention, which are described and/or claimed below. This discussion is believed to be helpful in providing the reader with background information to facilitate a better understanding of various aspects of the present invention. Accordingly, it should be understood that these statements are to be read in this light, and not as admissions of prior art.

The Center for Disease Control and Prevention (CDC) estimates that 48 million people get sick from foodborne illness, 128,000 are hospitalized and 3,000 die every year. One in six Americans get sick from foodborne illness every year. Foodborne illnesses are a major concern in modern society with estimates from the U.S. attributing 51% of these illnesses to plants and 42% to land animals (see FIG. 1A) [see Painter, J. A., et al., Emerging Infectious Diseases, 2013, 19 (3), 407-415]. Further analyses of these estimates point to bacteria and viruses as the most common causes of these illnesses (see FIG. 1B).

Of the 48 million people that get ill every year from foodborne agents, only 9 million of these are due to known pathogens. Yet, even when the contaminants are known, pathogenic detection methods—although reliable—are slow and time-consuming: The best detection methods today take anywhere from 2-7 days due to a need for enrichment [see Leonard, P. et al., Enzyme and Microbial Technology, 2003, 32 (1), 3-13; Pearson, B. et al., Food Microbiology, 2018, 72, 89-97]. This detection issue is exacerbated by the fact that food producers, who may lack food safety expertise, are the main source of contamination even though the food supply chain is extensive [Pearson, B. et al., Food Microbiology, 2018, 72, 89-97].

The U.S. Food and Drug Administration (FDA) recognizes Norovirus, Salmonella typhi, Escherichia coli O157:H7 or Shiga toxin-producing E. coli, Shigella spp. and Hepatitis A virus as the “big 5” causes for foodborne illness [see Fda.gov. (2018). Retail Food Protection: Employee Health and Personal Hygiene Handbook]. Early and rapid detection of such disease-causing microorganisms thus becomes important to ensure food safety. And so, biosensors have been used for the detection of pathogens. Biosensors are and have been of interest in the food safety, clinical medicine, environmental monitoring, and defense sectors for a long time. As can be seen from FIG. 2, a large number of biosensors have been tested and mostly designed for a narrow scope of operating conditions. Various biosensors with varied detection mechanisms have been used but only a few have stood the test of time. Although many sensors have been used on an ad-hoc basis for specific issues, not many have the potential for commercialization or being used for on-field measurements, which is an area of concern.

A number of publications can be found for biosensors which sense specific microorganisms in food and water. Typically, these biosensors are based on principles of standard plate count, flow cytometry, bioluminescence, optical sensing, or electrochemical biosensing [see Ivnitski, D. et al., Biosensors for Detection of Pathogenic Bacteria, Biosensors and Bioelectronics 1999, 14 (7), 599-624; Salzman, G. et al., Light scattering and cytometry. In: Melamed, M. R., Lindmo, T., Mendelsohn, M. L. (Eds.) Flow cytometry and sorting. John Wiley, New York, 1990, pp. 105-153; Hejris, B. et al., Optical, on-Line Bacteria Sensor for Monitoring Drinking Water Quality. Scientific Reports 2016, 6 (1); and Kim, H.-J. et al., A Novel Liposome-Based Electrochemical Biosensor for the Detection of Haemolytic Microorganisms. Biotechnology Techniques 1995, 9 (6), 389-394]. Of these different types, the electrochemical biosensors based on amperometric detection rely on a heterogeneous process of electron transfer, and so electrochemical measurements can be made at the electrode surface even with small volumes of sample [see Ivnitski, D. et al., Biosensors for Detection of Pathogenic Bacteria, Biosensors and Bioelectronics 1999, 14 (7), 599-624]. Electrochemical biosensors employ a wide variety of synthetic techniques and electroanalytical measurements to obtain selective, sensitive, and rapid detection. Current electrochemical biosensors rely on the attachment of labels (usually enzymes), or the interaction of bioreceptors and bacterial cells, which can alter the electrical parameters like current, potential, or impedance at the surface of electrodes [see M. Xu, R. Wang, Y. Li Electrochemical biosensors for rapid detection of Escherichia coli O157:H7, Talanta, 162 (2017) 511-522]. The first class of electrochemical biosensors are known as label-dependent, while the second class are label-independent.

In label-dependent biosensors, an electrocatalyst is designed to measure the concentration of an active analyte in the solution (sample). Typically, the analyte is not present in the initial solution. A label, typically an enzyme, in the presence of the microorganism produces the analyte. The concentration of analyte produced is a function of the concentration of micro-organisms present in the sample. As the concentration of analyte increases, the electrocatalyst is able to develop a response (current and/or voltage) that is measured and related to the concentration of microbes.

In summary, the label (commonly enzyme) accelerates the electrochemical active analyte in the solution to transfer electrons to the electrode. For example, a liposome-based amperometric biosensor (the current is measured when a constant potential is applied) had the potential to detect concentrations of different strains of E. coli [see Kim, H.-J.; Bennetto, H. P.; Halablab, M. A. A Novel Liposome-Based Electrochemical Biosensor for the Detection of Haemolytic Microorganisms. Biotechnology Techniques 1995, 9 (6), 389-394]. Several groups have reported the measurement of intracellular enzymes present in E. coli—enzymes such as β-D-glucuronidase (GUS) and β-D-galactosidase (Gal) extracted through enzyme induction, and reactions with various substrates on enzymes form electroactive products that can be measured and correlated to varying concentrations of bacteria using amperometric and potentiometric techniques (the potential is measured when a constant current is applied) [see Wutor, V. C. et al., A Novel Biosensor for the Detection and Monitoring of β-d-Galactosidase of Faecal Origin in Water. Enzyme and Microbial Technology 2007, 40 (6), 1512-1517; Rochelet, M. et al., Rapid Amperometric Detection of Escherichia Coli in Wastewater by Measuring β-D Glucuronidase Activity with Disposable Carbon Sensors. Analytica Chimica Acta 2015, 892, 160-166; Noh, S. et al., Facile Electrochemical Detection of Escherichia Coli Using Redox Cycling of the Product Generated by the Intracellular β-d-Galactosidase. Sensors and Actuators B: Chemical 2015, 209, 951-956; Chen, J. et al., Electrochemical Nanoparticle-enzyme Sensors for Screening Bacterial Contamination in Drinking Water. The Analyst 2015, 140 (15), 4991-4996; and Geng, P. et al., A DNA Sequence-Specific Electrochemical Biosensor Based on Alginic Acid-Coated Cobalt Magnetic Beads for the Detection of E. coli. Biosensors and Bioelectronics 2011, 26 (7), 3325-3330]. However, the performance of the amperometric biosensor is restricted to: (i) activation/kinetics (time required in the culture to produce the analyte), (ii) interference of other electrochemical compounds that have similar redox potentials to the analyte, and (iii) the stability of the enzyme at the applied potential [see M. Xu, R. Wang, Y. Li Electrochemical biosensors for rapid detection of Escherichia coli O157:H7, Talanta, 162 (2017) 511-522]. In summary, a short time response is not feasible with this type of sensor as it requires time to produce the analyte.

The label-independent approach measures the resistance and/or conductivity of the solution, or analyzes electron transfer at the surface of the electrode, which can be measured by electrochemical impedance spectroscopy (EIS) [see M. Xu, R. Wang, Y. Li Electrochemical biosensors for rapid detection of Escherichia coli O157:H7, Talanta, 162 (2017) 511-522]. This type of approach is known as involving the use of impedometric sensors. The technique applies a sinusoidal potential with a small amplitude to the electrochemical system and measures the resulting current over a range of varying excitation frequencies. The data that is obtained is fitted into an equivalent electric circuit that is correlated to the concentration of the microorganisms. The key to this method is to immobilize the micro-organisms at the surface of the electrode or the bioreceptors. For example, some groups have reported the use of impedimetric sensors where a substrate is chemically adsorbed onto an electrode surface (typically gold) to allow microorganisms to bind to the electrode, creating a complex electrode-solution interface [see Li, Y. et al., Impedance Based Detection of Pathogenic E. coli O157:H7 Using a Ferrocene-Antimicrobial Peptide Modified Biosensor. Biosensors and Bioelectronics 2014, 58, 193-199; Liu, X. et al., Biosensors Based on Modularly Designed Synthetic Peptides for Recognition, Detection and Live/Dead Differentiation of Pathogenic Bacteria. Biosensors and Bioelectronics 2016, 80, 9-16; and Geng, P. et al., Self-Assembled Monolayers-Based Immunosensor for Detection of Escherichia Coli Using Electrochemical Impedance Spectroscopy. Electrochimica Acta 2008, 53 (14), 4663-4668]. The advantage of the label-independent approach is that an immediate measurement of the microorganisms can be achieved. However, these methods are limited by the immobilization procedures which can significantly affect the reproducibility and regenerability of the fabricated sensors. In addition, the limit of detection using EIS for pathogen detection is still not low enough [see O. Lazcka, F. J. D. Campo, F. X. Munoz, Pathogen detection: a perspective of traditional methods and biosensors, Biosens. Bioelectron. 22 (2007) 1205-1217].

Label-dependent biosensors have high specificity and sensitivity, but cannot provide results in real time. Label-independent sensors, on the other hand, allow for real time sensing, but are not so specific to live cells. In summary, electrochemical biosensors provide advantages for miniaturization, ease of integration for online measurement of bacteria in water and food. The continuous response of an electrochemical system allows for online control and the equipment required for electrochemical systems are simple and cheap compared to most other systems. However, the complexity of the synthetic procedures (substrate synthesis, electrode modifications), and complex analytical techniques (cell lysing, enzyme extraction), limits the practicality of these technologies on a more global scale. Ideally an electrochemical biosensor that combines the advantages of the label-independent with the detection limit of the label-dependent, utilizing relatively cheap materials, and simplified electrode configuration, would advance the practicality and feasibility of electrochemical biosensors for E. coli and other pathogens detection in food.

### SUMMARY OF THE INVENTION

Certain exemplary aspects of the invention are set forth below. It should be understood that these aspects are presented merely to provide the reader with a brief summary of certain forms the invention might take and that these aspects are not intended to limit the scope of the invention. Indeed, the invention may encompass a variety of aspects that may not be explicitly set forth below.

To address the aforementioned issues, aspects of the present invention include an electrochemical microbial sensor (EMS) that combines the advantages of label-dependent and label-independent electrochemical biosensors. Among other uses, the EMS may be used for the accurate detection of different pathogens in food.

And so, an aspect of the present invention may provide an electrochemical sensor, including a working electrode, a reference electrode, and a counter electrode. The working electrode may include a transition metal, and is contacted with a solution including an alkaline media for oxidation of the transition metal, such that the sensor may be used to provide data to quantify the amount of a pathogen in the solution. In certain embodiments, the transition metal of the working electrode is nickel. And, in certain embodiments, the working electrode may be a rotating disk electrode, wherein the working electrode rotates in a solution including an alkaline media.

Another aspect of the present invention may provide an electrochemical sensor, including a working electrode, a reference electrode, and a counter electrode. The working electrode may include a transition metal or combinations of transition metals, and graphene, and is contacted with a solution including an alkaline media for oxidation of the transition metal, such that the sensor may be used to provide data to quantify the amount of a pathogen in the solution. In certain embodiments, the transition metal of the working electrode is nickel, and the working electrode includes graphene-layered nickel. And, in certain embodiments, the working electrode may be a rotating disk electrode, wherein the working electrode rotates in a solution including an alkaline media

### BRIEF DESCRIPTION OF THE DRAWINGS

The accompanying drawings, which are incorporated in and constitute a part of this specification, illustrate embodiments of the invention and, together with the general description of the invention given above and the detailed description of the embodiments given below, serve to explain the principles of the present invention.

FIG. 1A is a graph showing attribution of foodborne illnesses to food commodities differentiated into the individual food commodities, and FIG. 1B is a graph showing the causes in each food commodity.

FIG. 2 is a chart showing the types of biosensors applied in several fields of engineering (with the biosensors being classified based on their principle of detection).

FIG. 3 is a view of an embodiment of a three-electrode configuration of an EMS probe in accordance with principles of aspects of the present invention. Ni is used as the working electrode (WE) while Pt is used as the reference electrode (RE) and counter electrode (CE) in this embodiment.

FIG. 4 is a graph based on the procedure for the formation of the electrocatalyst, activation step. CV is performed at 15 mV/s for 5 in 1 M KOH. Sustained periodic state is achieved after 5 cycles.

FIGS. 5A-5D are graphs showing results from the testing step of the EMS and calibration curves. All the experiments are performed using 0.01 M KOH as the electrolyte. FIG. 5A shows chronoamperometry current profiles of low concentration E. coli in water/electrolyte. FIG. 5B shows calibration curve and mathematical equation for measuring E. coli of low concentrations in water. FIG. 5C shows chronoamperometry current profiles of high concentration E. coli in water/electrolyte. And FIG. 5D shows calibration curve and mathematical equation for measuring E. coli of high concentrations in water.

FIG. 6A shows a methodology implemented for the development of the EMS-G1. And FIG. 6B shows the overall methodology of the EMS-G1 for pilot testing in a municipal wastewater treatment plant in India.

FIGS. 7A and 7B are representations of electrode/electrolyte interface in the absence (FIG. 7A) and presence (FIG. 7B) of E coli in a RDE. Integration of nanoelectrode architectures are also represented.

FIGS. 8A and 8B are cyclic voltammograms showing higher currents associated with the formation of NiOOH in alkaline media in different nanoelectrode architectures at 10 mV/s scan rates. FIG. 8A shows graphene layer Ni electrode compared to Ni foil in 0.1 M KOH solution when scanned between 0.2 to 0.7V vs. Hg/HgO reference electrode. And FIG. 8B shows ERGO-Ni nanocomposite electrode and ERGO electrode in 1M KOH solution.

FIG. 9 is a schematic representation of the tasks involved in this project for achieving E. coli detection in raw vegetables such as lettuce and spinach.

FIG. 10 is a schematic representation of the electrode/electrolyte interface of graphene-layered electrodes. The additional graphene layer enhances the Helmholtz contribution thereby increasing the concentration of charges at the interface leading to an increased rate of reaction.

### DETAILED DESCRIPTION OF THE INVENTION

One or more specific embodiments of the present invention will be described below. In an effort to provide a concise description of these embodiments, all features of an actual implementation may not be described in the specification. It should be appreciated that in the development of any such actual implementation, as in any engineering or design project, numerous implementation-specific decisions must be made to achieve the developers' specific goals, such as compliance with system-related and business-related constraints, which may vary from one implementation to another. Moreover, it should be appreciated that such a development effort might be complex and time consuming, but would nevertheless be a routine undertaking of design, fabrication, and manufacture for those of ordinary skill having the benefit of this disclosure.

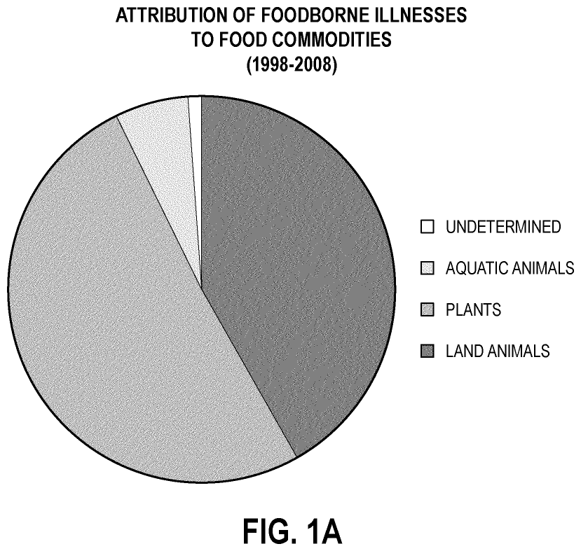

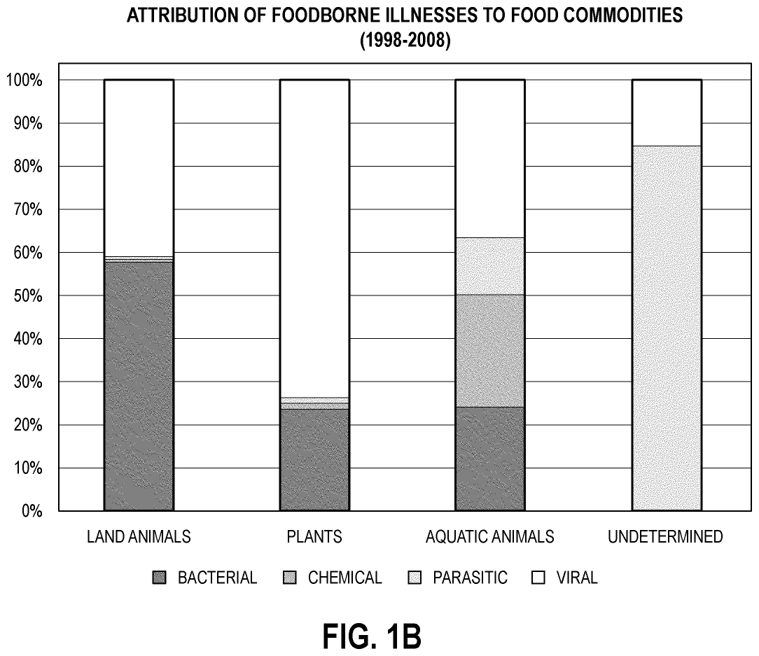

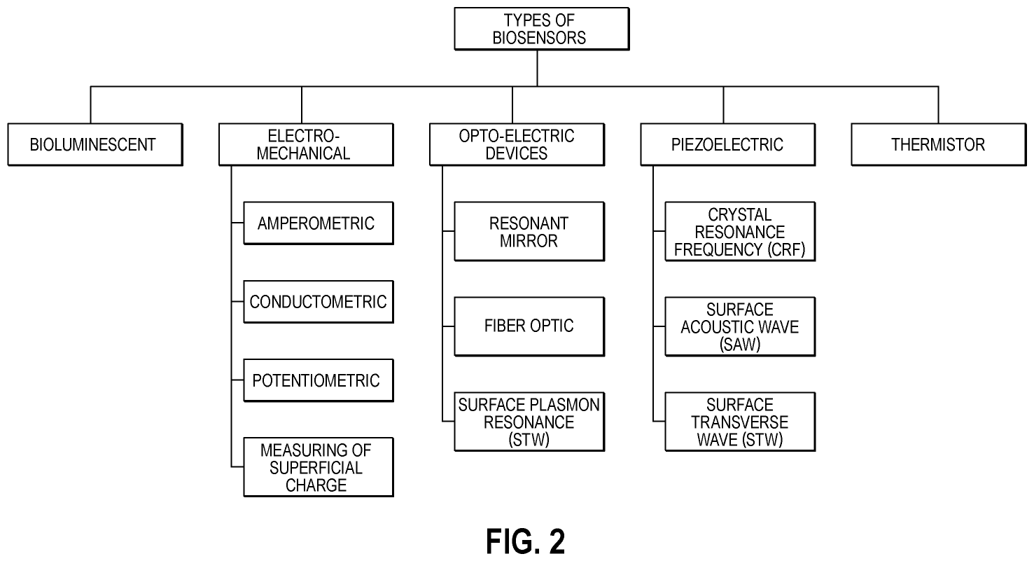

To address the aforementioned issues, aspects of the present invention include an electrochemical microbial sensor (EMS) that combines the advantages of label-dependent and label-independent electrochemical biosensors. Among other uses, the EMS may be used for the accurate detection of different pathogens in food. As will be described in greater detail below, the EMS may be used for detection of foodborne pathogens, primarily E. coli in raw vegetables such as lettuce/spinach. The focus on E. coli in vegetables is important, as 51% of foodborne illnesses come from plants (FIG. 1B), as described above. Thus, aspects of the invention include the integration of nanotechnology for the development of advanced electrode architectures that have the potential to increase the sensitivity limit of the EMS. The EMS disclosed herein: (1) optimizes the electrocatalyst structure and composition to improve the detection limit of E. coli in water; (2) implements the electrocatalyst into the current methodology and its extension for E. coli measurement in food; and (3) evaluates parameters such as detection limit and durability of the sensor. The new electrodes described herein for enhancing the current sensitivity are not only able to detect the E. coli viability but also sense concentrations as low as 0 cfu/g (no E. coli). Further, the EMS is easier to integrate in an actual field environment.

To that end, the present inventors have demonstrated (and describe herein) an embodiment of the EMS for the quantification of E. coli at levels that are found in a typical wastewater treatment plant (a first generation EMS; a first embodiment). A portable EMS has been demonstrated with a response time for the concentration of E coli in water of less than 5 minutes and an experimental uncertainty of 2-10% when compared with the standard plate count (SPC) for enumeration of bacteria. This detection limit would need to be improved for its application in the detection of pathogens in food. And, below, a second generation EMS (a second embodiment) is described that includes such an improved detection limit.

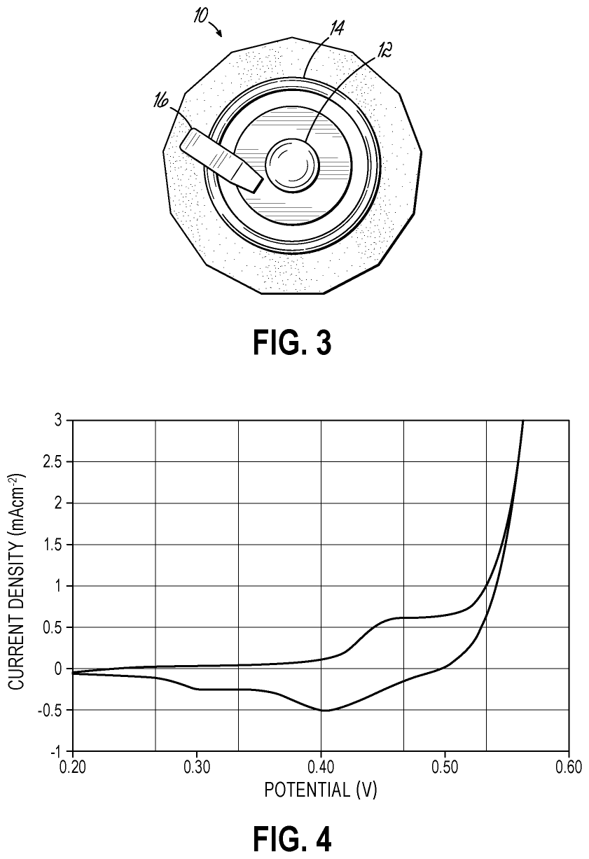

The EMS may be an amperometric sensor that utilizes the constant potential oxidation of nickel hydroxide (Ni(OH)₂) (or other potential transition metals, such as Co) to nickel oxyhydroxide (NiOOH) on a rotating disk electrode (RDE) in alkaline media to quantify E. coli in synthetic solutions ranging from 10²-10¹⁰ colony forming units per milliliter (CFU/mL). The RDE technique is applied using a small size electrode (e.g., 5 mm diameter) to introduce controlled, consistent mass transport of hydroxyl ions and E. coli to the surface of the Ni electrode and to provide a uniform current distribution on the electrode, providing some insight into the detection mechanism for the detection process. FIG. 3 shows the configuration of an EMS 10 in accordance with principles of aspects of the present invention, the EMS 10 including a working electrode (WE) 12, a reference electrode (RE) 14, and a counter electrode (CE) 16. In one embodiment, nickel foil was chosen as the working electrode (WE), while Pt was used as the quasi-reference electrode and counter electrode, respectively.

In certain embodiments, nickel may be used due to its ability to form a redox couple when placed in alkaline media and an electric field is applied in a certain potential window as represented in Equation 1 (Eq. 1): Ni(OH)₂+OH⁻⇄NiOOH+e⁻

One advantage of the embodiment of EMS that includes nickel (over previous electrochemical biosensors) lies in the fact that the nickel oxyhydroxide (NiOOH) electrocatalyst can be generated locally (in-situ) at the electrode surface as and when required. Hence, it eliminates the complications in design of enzymatic biosensors where there is always a potential threat that the inactivation of enzymes could hinder the sensing process. FIG. 4 presents the electrochemical procedure that is implemented for the generation of the NiOOH electrocatalyst. The Ni(OH)₂ catalyst layer is formed using cyclic voltammetry (CV) in an alkaline environment containing 1 M KOH. The CV procedure is performed in a potential window of 0.20-0.57 V vs. Pt at a scan rate of 15 mV/s. The sustained periodic cycle, shown in FIG. 4, is achieved after 5 cycles in accordance with previously reported results by one of the present inventors [see V. Vedharathinam, G. G. Botte, Direct evidence of the mechanism for the electro-oxidation of urea on Ni(OH)2 catalyst in alkaline medium, Electrochimica Acta, 108 2013, 660-665]. The anodic peak at 0.46V vs. Pt is attributed to the one-electron oxidation of Ni(OH)₂ to form NiOOH (Eq. 1). At potentials higher than 0.52 V vs. Pt, the oxidation of water in alkaline media starts to take place. During the reverse scan, two different cathodic peaks are observed, which are attributed to the formation of two different states of Ni(OH)2 [see V. Vedharathinam, G. G. Botte, Direct evidence of the mechanism for the electro-oxidation of urea on Ni(OH)₂ catalyst in alkaline medium, Electrochimica Acta, 108 2013, 660-665]. The first cathodic peak at 0.40 V vs. Pt represents the one-electron reduction of β-NiOOH to form β-Ni(OH)₂ and the second, smaller cathodic peak at 0.30 V vs. Pt represents the one-electron reduction of γ-NiOOH to form α-Ni(OH)₂. The formation of the electrocatalyst is the first step of the EMS methodology and may be referred to herein as the “activation step.” This first step (activation step) may take approximately 4 minutes in certain embodiments.

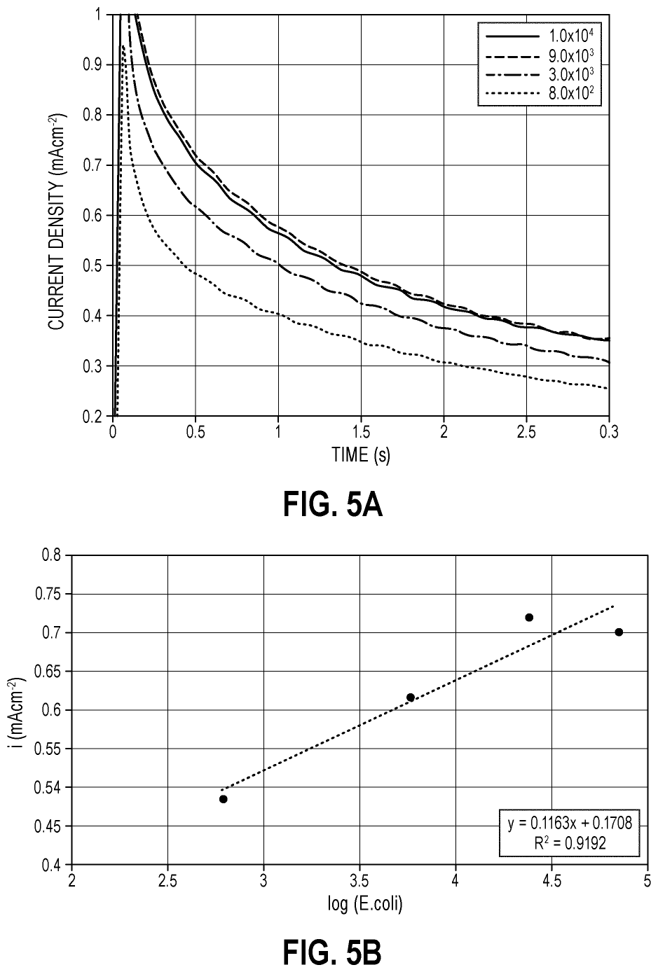

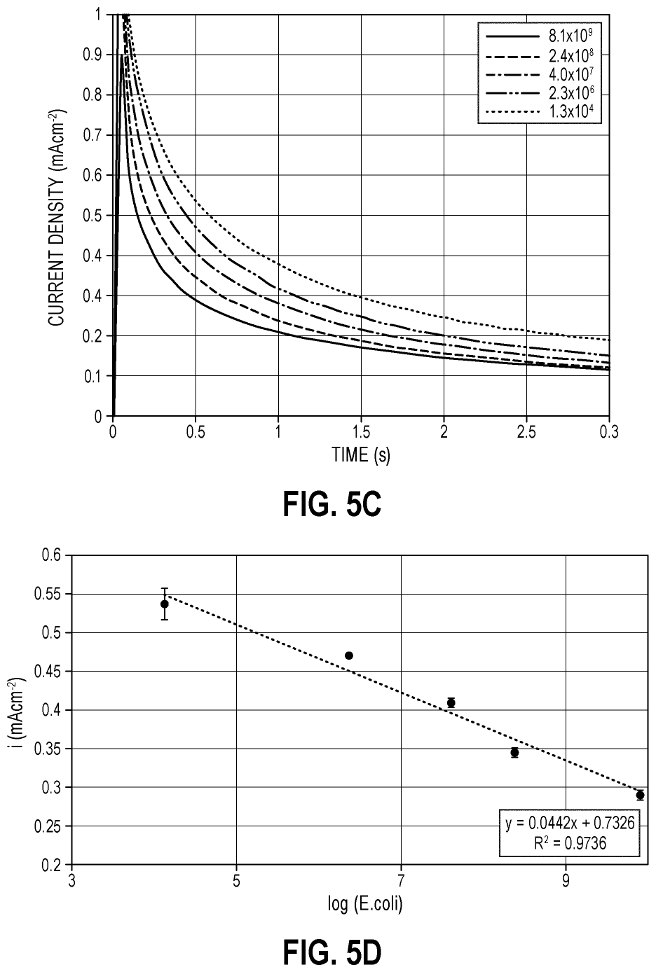

After the activation step, the electrode is then immersed in a solution containing a 0.01 M KOH solution and rotated for the “testing/sensing step,” The solution may include a pathogen that is being tested for (such as E. coli). And, in testing the EMS described herein (to demonstrate proof-of-concept), the electrode was immersed in a solution containing E. coli and a 0.01 M KOH solution and rotated at 1600 rpm (optimization of the rotation of the electrode was performed for the embodiments of the present electrode design). The open circuit potential (OCP) may be monitored until reaching a steady value (60 seconds), indicating the surface Ni(OH)₂/NiOOH layer is in equilibrium with solution. Chronoamperometry may then be used to measure the constant potential nickel oxidation and reduction reactions. The Ni(OH)₂ is oxidized at 0.58 V vs. Pt for 5 seconds to provide sufficient overpotential for NiOOH formation while avoiding excessive water electrolysis. Finally, the NiOOH is reduced at 0.10 V vs. Pt for 15 seconds to reform the Ni(OH)₂ layer prior to subsequent testing. It should be noted that the presence of 0.01 M KOH and the electrochemical reaction conditions cause no significant variation in the viability of E. coli, indicating no cell death nor cell growth occurs over the course of the testing/sensing procedure. In demonstrating the present EMS, the testing procedure was repeated three times and the average current at 0.5 s was calculated and calibrated with respect to the E. coli concentration (determined by SPC). Calibration curves were obtained with a widely used, non-pathogenic, laboratory strain of E. coli (DH5α). A mathematical correlation was applied to relate the current with the concentration of E. coli in the solution. Results of the different experiments and the mathematical correlations for the sensor are presented in FIG. 5. This “testing/sensing step” only takes approximately 4 minutes in certain embodiments.

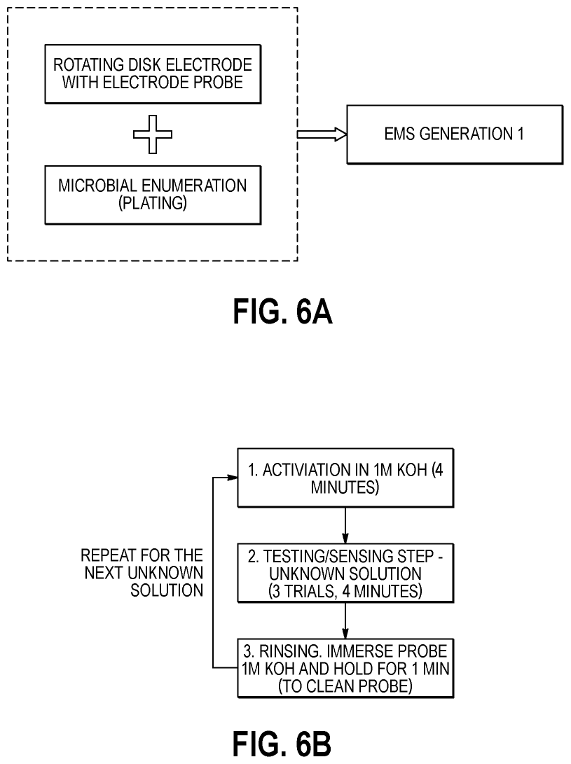

Finally, the sensing probe is rinsed in 1M KOH for cleaning purposes. This procedure disinfects the probe from residual E. coli contamination while also helping to close a cycle for new measurements. This last step in the procedure “rinsing step” takes approximately 1 minute. The methodology that was used for the development of the EMS generation 1 (EMS-G1)—the first embodiment of the EMS—is shown in FIG. 6A. As indicated, electrochemical methods—controlled conditions of the electrochemical probe in a rotating disk electrode system (RDE)—combined with microbiology—microbial enumeration via plating—were implemented to develop the EMS methodology. The methodology was implemented into an online EMS-G1 that can be controlled and operated remotely. And, the EMS-G1 was pilot tested in a facility in Goa, India. That pilot test gave the results shown below in Table 1. A similar pilot test was performed at a waster water treatment plant in Athens, Ohio. That pilot test gave the results shown below in Table 2.

TABLE 1
Field Test at BITS, Goa, India (August 2018)

| | Concentration | Concentration | |
|---|---|---|---|
| Unknown/ | from Standard | from EMS | % error |
| Sample | Plating (CFU/ml) | (CFU/ml) | (log scale) |
| 1 | 10400 | 3552 | 11.62 |

TABLE 2
Field test at Waste water treatment plant (sludge
2% solids in water), Athens, Ohio, USA (March 2019)

| | Concentration | Concentration | | |
|---|---|---|---|---|
| | for Total | for E. coli | | |
| | Coliform from | from | Concentration | |
| Unknown/ | Commercial | Commercial | from EMS | % error |
| Sample | lab (MPN/ml)* | lab (MPN/ml)* | (CFU/ml) | (log scale) |
| 1 | 51720 | 1340 | 793 | 7.30 |

*Masi ® Environmental Laboratory (Commercial Lab)

The overall methodology for sensing in the EMS-G1 is shown in FIG. 6B: 1. Activation, 2. Sensing, and 3. Rinsing. Overall, time for the whole process is less than 15 minutes (including electrocatalyst formation, while sensing time is only 4 minutes).

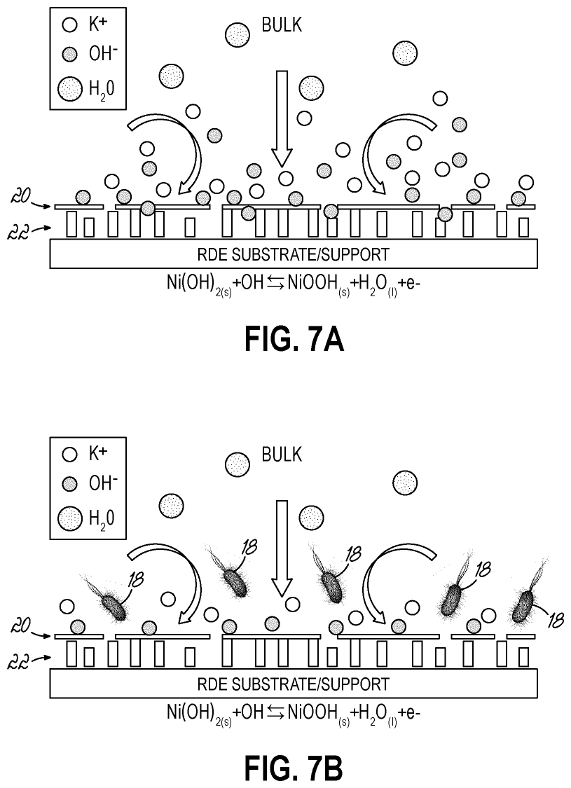

The EMS described herein works on the basis of chronoamperometry detection with combined advantages of label-dependent (formation/activation of catalyst) and label-independent (effects are present at high concentrations of E. coli). In the case of EMS where nickel oxyhydroxide formed in Equation 1 is the electrocatalyst, the electron is donated at the anode as a result of the forward reaction of Equation 1 (oxidation) and electron is accepted at the cathode for the catalyst to be reduced as shown in the reverse reaction of Equation 1. Bacterial cells generate energy using a process called the electron transport chain (ETC). During the ETC electrons move from a donor to a receptor, via a series of intermediates, and in the process, protons are pumped from the inside of the cell to the outside [see Henkel, S. G. et al., Basic Regulatory Principles of Escherichia coli's Electron Transport Chain for Varying Oxygen Conditions. PLoS ONE 2014, 9 (9), e107640]. The resulting imbalance in proton distribution across the cellular membrane is called the proton motive force (PMF) and is used by bacterial cells to generate energy. Without being bound to any theory, it is believed that the mechanism through which the EMS functions is by detecting an interaction between the protons outside the bacterial membrane and ions generated at the anode/cathode thereby resulting in an increase in current with increasing concentration of E. coli at relatively low concentration (<10⁴ cfu/ml). This can be seen in the chronoamperometry plot shown in FIG. 5A and its corresponding calibration curve represented in FIG. 5B. On the contrary, as the concentration of E. coli increases (>10⁴ cfu/ml), the electron transport chain of such a large number of E. coli causes a steric hindrance to the nickel oxidation reaction taking place at the anode resulting in a drop in the current. The chronoamperometry plot for E. coli in water at high concentrations can be seen in FIG. 5C and its corresponding calibration curve is represented in FIG. 5D. FIG. 7 shows a schematic of the mechanisms hypothesized to explain the operation of the EMS at low and high concentrations of E coli in the electrolyte solution and its interaction with the electrode (electrode/electrolyte/E coli interface), including a layered graphene electrode 20 and nanocatalysts/catalysts 22.

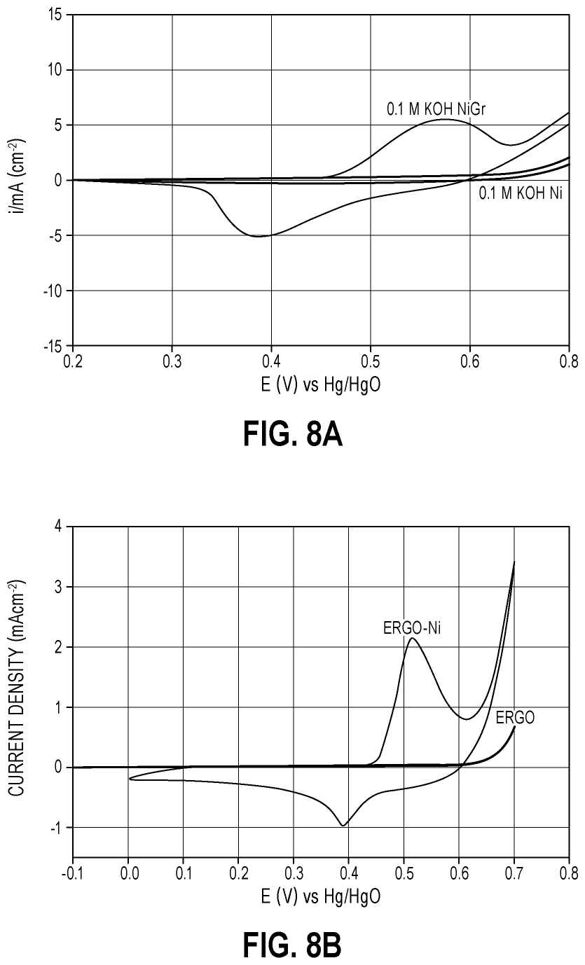

From the developments obtained from using EMS for E. coli detection in water, it is evident that, in the embodiment shown and described as generation 1, E. coli concentrations higher than 102 cfu/ml are able to be detected and quantified. But this number is still in the unsatisfactory range of E. coli for food samples according to the Center for Food Safety, Hong Kong. And so, another embodiment of the present invention includes an EMS having a detection limit of <20 cfu/g, which is considered as the satisfactory range of E. coli in raw vegetables. This is accomplished in the generation 2 embodiment by introducing nanostructured electrodes instead of the nickel foil (generation 1). Without being bound by any theory, it is believed that the introduction of such nanostructured electrodes, like graphene-layered nickel electrodes, would facilitate better current sensitivities and in turn improve the detection limit of the sensor. This increase in current sensitivity results from an increase in concentration of charges at the electrode/electrolyte interface. An evidence for this has been demonstrated by one of the present inventors: electrode architectures consisting of graphene-layered nickel (NiGr) and reduced graphene oxide nickel composites produced higher currents when compared to pure Ni electrode in KOH solutions of same concentration as depicted in FIGS. 8A and 8B, respectively [see Botte, G. G. Graphene Layered Electrodes. U.S. Patent Application Publication No. 20160251765A1; and Wang, D.; Yan, W.; Vijapur, S. H.; Botte, G. G. Electrochemically Reduced Graphene Oxide-nickel Nanocomposites for Urea Electrolysis. Electrochimica Acta 2013, 89, 732-736]. Previously, these nanocomposites were successful in enhancing the urea electro-oxidation current due to the large active surface areas of graphene sheets and the synergistic contribution of nickel and graphene sheets as it can be seen from FIG. 8 [see also Wang, D.; Yan, W.; Vijapur, S. H.; Botte, G. G. Electrochemically Reduced Graphene Oxide-nickel Nanocomposites for Urea Electrolysis. Electrochimica Acta 2013, 89, 732-736]. In a similar fashion these nanocomposite electrodes are expected to enhance the currents of the EMS probe facilitating better E. coli detection limits in food.

Some features of the EMS and methods described herein include (but are not limited to) fast detection (e.g., less than 0.5 s), no cultures needed, on-line probe, method and be extended for unattended, online. Alternative uses or aspects include microprobes, online sensor, automatic sensor, connected to WiFi for measurements online, biomedical, water, and/or food applications.

As described above, the most common method for quantifying E. coli (or bacterial concentration in general) is by the Standard Plate Count (SPC) technique [see also Gracias, K. S.; McKillip, J. L. A Review of Conventional Detection and Enumeration Methods for Pathogenic Bacteria in Food. Canadian Journal of Microbiology 2004, 50 (11), 883-890]. For this technique, the incubation time for growth of bacteria into individual countable colonies is between 24-120 hours depending on the bacterial species and culture medium used [see Lechevallier, M. W.; Seidler, R. J.; Evans, T. M. Enumeration and Characterization of Standard Plate Count Bacteria in Chlorinated and Raw Water Supplies. APPL. ENVIRON. MICROBIOL. 2018, 40, 9]. However, via the use of the EMS in accordance with the principles described herein, the sensing time may be reduced to as low as 300 seconds. This is at least 99.65% faster compared to SPC. Lately, a number of test kits have become available for measuring bacterial concentrations by recording the luminescence signals of samples. One such test kit is the BacTiter-Glo™ microbial cell viability assay. This kit works on the basis of an interaction between the BacTiter-Glo™ reagent and adenosine triphosphate (ATP) from the bacteria, resulting in a luminescent reaction. The luminescent signals are captured and recorded with a sensing time of 5 minutes [see Hammes, F.; Goldschmidt, F.; Vital, M.; Wang, Y.; Egli, T. Measurement and Interpretation of Microbial Adenosine Tri-Phosphate (ATP) in Aquatic Environments. Water Research 2010, 44 (13), 3915-3923]. This means that the EMS will be as rapid in sensing as the commercially available test kits and moreover, it has the edge over the test kits for its potential to be used directly in the field with minimal monitoring required.

As described above, various embodiments of the EMS include a generation 1 EMS (pure nickel foil working electrode) and a generation 2 EMS (graphene layered nickel/graphene oxide-nickel nanocomposite). It is anticipated that the generation 2 EMS will have better detection limit, sensitivity, and durability for detecting E. coli in raw vegetables as compared to the generation 1 embodiment. And so, the generation 2 embodiment may be more useful for food detection (as compared to the generation 1 embodiment)—though the generation 1 embodiment is still superior in use to previous detection apparatus and methods. A review article on different biosensors by Poltronieri et al. concludes that there are several issues such as pretreatment of sample, enrichment of bacteria in culture broth, proper storage of reagents, detection limit and sensitivity of probe that hinder the on-field integration of biosensors [see Poltronieri, P.; Mezzolla, V.; Primiceri, E.; Maruccio, G. Biosensors for the Detection of Food Pathogens. Foods 2014, 3 (3), 511-526]. The EMS addresses the problems of pretreatment of sample as the electrode being used in EMS doesn't require long hours of pretreatment and moreover the electrocatalyst for detection can be locally generated in-situ. The issue of enrichment of bacteria in culture broth is countered by increasing the pH of test solution for measurement of E. coli without actually killing them. There are no reagents needed for EMS measurements except for KOH which is the only solution required for overall measurement and hence the issues with proper storage and handling of reagents could be overcome. Furthermore, KOH is inexpensive therefore removing the need for costly reagents (such as with BacTiter-Glo™).

The overall goal of the Nanotechnology for Agricultural and Food Systems is to “develop nanotechnology enabled solutions for food and nutrition security through . . . enhanced food safety and biosecurity” and specifically the (1) development of nano-scale based sensing mechanisms for accurate, reliable and cost-effective early and rapid detection of pathogens; and (2) development of portable and field deployable sensors and devices for real-time detection and screening to identify targets requiring no additional laboratory analyses. The EMS embodiments described herein, and the nano-scale electrocatalyst (graphene-based electrode), projects behaviors that would not be observed at the large-scale by simply combining nickel with carbon. This enhanced activity increases the accuracy and reliability of the method developed. Furthermore, the use of an electrochemical system that is calibrated to the pathogen will provide a sensor that yields a single-read out i.e. a user-friendly technology that does not require additional laboratory analyses or field-specific knowledge.

Thus, the EMS embodiments described herein, including the EMS based on graphene nanotechnology, have the potential to be a highly sensitive, rapid, portable and user-friendly pathogenic sensing for food safety. These features provide and improvement on current techniques based on both detection limits and possession of pathogen-specific knowledge. This will assist in ensuring a sustainable approach through the safe satisfaction of human food and fiber needs across the agricultural food supply chain.

### EXAMPLES

The following are prophetic examples.

One objective of the development of the EMSs described herein demonstrate the Electrochemical Microbial Sensor (EMS) for detection of foodborne pathogens. To accomplish this objective, detection of E. coli in raw vegetables such as lettuce/spinach will be used as a model system. Specific objectives are: (1) Optimization of electrocatalyst structure and composition to improve the detection limit of E. coli in water; (2) Implementation of as developed electrocatalyst into the current methodology and its extension for E. coli measurement in food; (3) Evaluation of parameters such as detection limit and durability of the sensor.

These objectives will be accomplished by conducting five tasks (five examples) as outlined below.

1. Optimization and evaluation of graphene-layered super electrodes for E. coli detection in water

2. Optimization and evaluation of electrochemically reduced graphene oxide-nickel nanocomposites for E. coli detection in water

3. Extension of generation 1 EMS methodology for E. coli detection in food

4. Evaluation of the optimized electrodes for E. coli detection in food

5. Integration of the optimum electrode with current methodology for E. coli detection in food and optimization of detection limit and durability

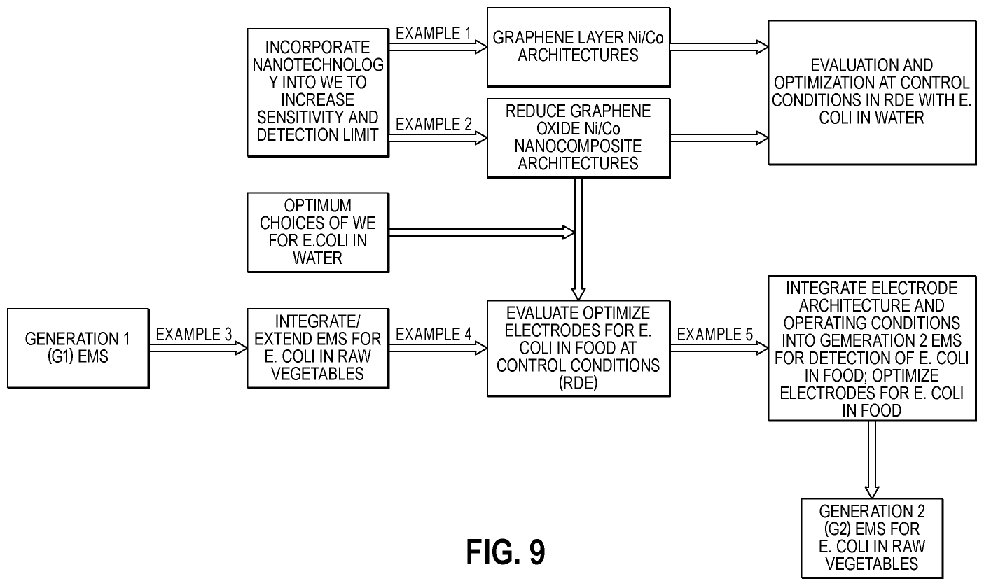

In general, the initial step will be to develop a set of electrodes which will be evaluated based on performance in water. Out of this set, the electrodes which perform better for detecting E. coli in water will be selected and the viability of their extension to detecting E. coli in food will be tested extensively. The results of these tests will be used for selecting the best electrode of the lot for detecting E. coli viability in food. Further, the process variables will be optimized to achieve enhanced detection limit, accuracy and durability of the sensor. An overview of the approach for methodology involved in this project is shown as a scheme in FIG. 9.

### Example 1. Optimization and Evaluation of Graphene-Layered Super Electrodes for E. coli Detection in Water

Introduction: Graphene-layered super electrodes typically consist of 1 to 5 layers of graphene coated on an active catalyst material in such a manner that at least a portion of the catalyst material is covered by the graphene layer [see U.S. Patent Application Publication No. 2016/0251765A1]. The graphene layer will be prepared by chemical vapor deposition (CVD) using electrolyzed coal as the carbon source, based on the experimental procedures described by Botte and Lu and Botte [see F. Lu, G. G. Botte, Ammonia Generation via a Graphene-Coated Nickel Catalyst, Coatings, 7 (2017), 1-11; and U.S. Patent Application Publication No. 2016/0251765A1]. The hypothesis behind using these graphene-layered electrodes is that they modify the electric double layer in such a way that the Helmholtz contribution is enhanced. This change increases the concentration of charges at the electrode/electrolyte interface which also implies an increased rate of reaction. An evidence for this can be seen in FIG. 8A. The idea behind using these graphene-layered electrodes is to improve the sensitivity of EMS for detecting very low concentrations of E. coli.

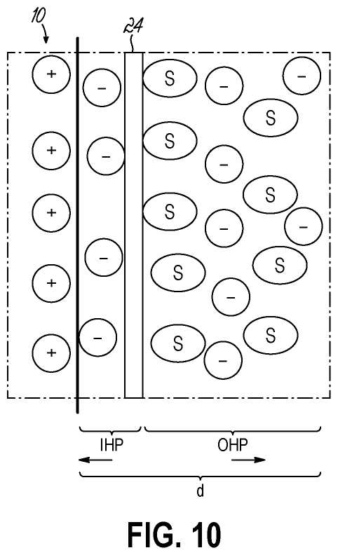

Approach: The approach for this task will be to prepare a set of graphene layered electrodes by changing the composition of underlying Ni-based active catalyst material and to evaluate their performance for E. coli detection in water with detection limit and durability as the check points. First, such electrode to be prepared will be an extension of the electrode being used in the current methodology. A layer of graphene prepared by CVD will be transferred onto to the surface of Ni foil to provide a larger surface to volume ratio of the working electrode and moreover, forming a third layer in the electrical double layer thereby increasing the concentration of ions near the electrode surface as discussed in the introduction of this task and also seen in FIG. 10. The next electrode will be graphene layer 24 coated onto an electrodeposited Ni electrode. The modification here is to deposit Ni on a Ni substrate before coating the graphene layer. The purpose of such an additional deposition is to increase the surface to volume ratio of the electrode thereby increasing the sensitivity of the sensor. Another modification to the electrode could be the introduction of cobalt to the complex. Co similar to Ni undergoes oxidation in alkaline media. Yan and Botte demonstrated a significant increase in the current density on electrodes consisting of Ni—Co prepared by electrodeposition in alkaline media [see W. Yan, D. Wang, G. G. Botte, Nickel and cobalt bimetallic hydroxide catalysts for urea electro-oxidation, Electrochimica Acta, 61 (2012), 25-30]. Typical composition ratios for an increase in the current density were reported in the range of 40:60, and 30:70 for Co:Ni [see W. Yan, D. Wang, G. G. Botte, Nickel and cobalt bimetallic hydroxide catalysts for urea electro-oxidation, Electrochimica Acta, 61 (2012), 25-30]. Hence, Co can be infused with Ni in the complex in different ratios before being coated with the graphene layer. The Co—Ni layers will be prepared by electrodeposition following the procedures described by Yan and Botte. Finally, a bare graphene layer coated on glassy carbon (GC) substrate as a control can be prepared and tested in a similar fashion as the other electrodes. The performance of all these electrodes for detection of E. coli in water will be tested under controlled conditions using a rotating disk electrode (RDE) setup, following the methodology described above and shown in FIG. 6A. The electrodes which are stable and can detect as low as 20 cfu/g E. coli (satisfactory number) will be chosen for further studies with food. Quantification of E. coli will be performed using the classical plating procedure.

The structural properties of the synthesized nanoparticle catalysts will be evaluated using a combination of X-ray diffraction (XRD), High-Resolution Transmission Electron Microscopy (HR-TEM), and Energy Dispersive X-ray Spectroscopy (EDS) for delineation of crystal structure, morphology and metallic composition, respectively. The surface area of the catalysts synthesized will be determined by the Brunauer-Emmett-Teller (BET) method. The electrochemical surface area (ESA) or active surface area of the catalysts will be measured from the surface coverage of hydrogen atoms (adsorbed and desorbed) on the catalyst during cyclic voltammetry. The loading of the metals such as Ni, Co, and Ni—Co will be kept in a range lower than 1 mg/cm², as significant current densities have been observed within this range [see W. Yan, D. Wang, G. G. Botte, Nickel and cobalt bimetallic hydroxide catalysts for urea electro-oxidation, Electrochimica Acta, 61 (2012), 25-30]. Operating variables related to the methodology will include the rotation rate of the RDE and the applied potential. The temperature of the solution will be kept constant and will be part of the SOP developed for the process. Temperature controller will be included in the EMS. It is envisioned that the temperature of the system will be kept at the range that will not disturb the concentration of E. coli, between 20-25° C.

Expected Results: The optimal operating conditions to develop the Ni/Ni—Co based graphene layered electrodes which have high sensitivity in the satisfactory and borderline ranges of E. coli will be obtained. Further, in Example 4 (below), these electrodes will be tested for being extended to detect the E. coli present in food.

Additionally, we will test another set of electrodes which are electrochemically reduced graphene oxide (ERGO)-nickel nanocomposites which lays the foundation for Example 2.

### Example 2. Optimization and Evaluation of Electrochemically Reduced Graphene Oxide-Nickel Nanocomposites for E. coli Detection in Water

Graphene based metal nanocomposites have been used as electrodes in sensors for their large active surface areas and improved electron transport [see Shan, C.; Yang, H.; Han, D.; Zhang, Q.; Ivaska, A.; Niu, L. Graphene/AuNPs/Chitosan Nanocomposites Film for Glucose Biosensing. Biosensors and Bioelectronics 2010, 25 (5), 1070-1074]. Electrodes such as PtNi nanoparticle-graphene composite have been successfully used for non-enzymatic amperometric detection of glucose [see Gao, H.; Xiao, F.; Ching, C. B.; Duan, H. One-Step Electrochemical Synthesis of PtNi Nanoparticle-Graphene Nanocomposites for Nonenzymatic Amperometric Glucose Detection. ACS Applied Materials & Interfaces 2011, 3 (8), 3049-3057]. These references could be used to synthesize ERGO/metal nanocomposites and test them for E. coli detection.

Approach: The possibility for using electrodes like ERGO-Ni, ERGO-Co, ERGO-Ni/Co (different ratios of Ni and Co) nanocomposites for non-enzymatic amperometric detection of E. coli in water will be investigated. Our group has already synthesized ERGO-Ni nanocomposite for urea electrolysis and it can be seen from the cyclic voltammetry curves of FIG. 8B of preliminary results section that there is a considerable increase in the current density of ERGO-Ni nanocomposite compared to bare ERGO showing potential that these electrodes could be used for detecting E. coli in lower concentrations. The same procedure will be implemented for ERGO-Ni synthesis for this case. Similarly, Ni can be substituted entirely or partially to form ERGO-Co and ERGO-Ni/Co nanocomposites respectively. The composition of Ni and Co in the latter complex can be modified and optimized for better sensitivity. Another electrode with glassy carbon as substrate can be the bare electrode for control. All the testing will be performed with an RDE setup under controlled conditions. Characterization of the electrodes and methods for the detection of E. coli as described in Example 1 will be followed.

Expected Results: The optimal operating conditions to develop the ERGO-metal nanocomposite electrodes which have high sensitivity in the satisfactory and borderline ranges of E. coli will be obtained. Further, in Example 4, these electrodes will be tested for being extended to detect E. coli present in food.

We will also use the current available method for sensing E. coli in water and try to extend it to work as such for E. coli detection in food. This is the basis for Example 3.

### Example 3. Extension of Generation 1 EMS Methodology for E. coli Detection in Food

The feasibility of implementing generation 1 EMS methodology with Ni foil as the working electrode directly for detection of E. coli in raw vegetables can be investigated. Leafy vegetables such as lettuce and spinach can be used as models for these tests.

Approach: In this Example, we will develop a standard operating procedure (SOP) for the detection of E. coli in leafy green vegetables using the generation 1 EMS. Spinach leaves will be sterilized by exposure to UV irradiation and subsequently inoculated with known amounts of E. coli. The amount of E. coli used to inoculate the leaves will range from very low concentrations (20 cfu/g) to high concentrations (108 cfu/g). Once the E. coli has dried onto the leaves each set will be tested for microbial contamination, using the EMS, as follows. 50 g of spinach leaves will be mixed with 450 ml of Butterfield's phosphate buffer water (diluent) in a mixing bag and homogenized using a Stomacher machine. Following homogenization, samples will be filtered to remove leaves, and the concentration of bacteria in the resulting liquid determined using the generation 1 EMS (following our established procedure). The exact concentration of E. coli present in each sample will be determined by SPC and compared to the experimentally determined concentration from the EMS. Once the procedure has been established for E. coli using spinach leaves, we will repeat the procedure for additional food borne pathogens (Salmonella typhimurium and Listeria monocytogenes) using various sources of leafy green vegetables (lettuce, green onions, cabbage) to determine how broad the range of detection is using the generation 1 EMS. These experiments can be performed simultaneously alongside Examples 1 and 2 with an added motive that at the end of Examples 1 and 2, developed electrodes could replace the Ni foil electrode of EMS and be used for E. coli detection in food with the standard operating procedure optimized from Example 3.

Expected Results: Formulation of a standard operating procedure for detection of food borne bacteria in leafy green vegetables using the current EMS methodology. As mentioned above, this SOP could be used in Example 4 by replacing Ni foil with electrodes chosen as a result of optimization from Examples 1 and 2 to achieve better sensing.

Further, alternate diluents which are compatible with alkaline media may be used.

### Example 4. Evaluation of Optimized Electrodes for E. coli Detection in Food

The results of Examples 1 and 2 are inevitable for classifying the electrodes tested based on the Center for Food Safety, Hong Kong standards for E. coli in ready-to-eat (RTE) foods. According to this standard, there are three levels of E. coli in RTE foods: satisfactory (<20 cfu/g), borderline range (20-10² cfu/g) and unsatisfactory (>10² cfu/g). In a similar fashion to this, we can classify the electrodes tested in Examples 1 and 2 into three levels based on their detection limit. Corresponding to the level of E. coli concentration, the synthesized electrodes could be used for testing E. coli in food using the standard operating procedure formulated from Example 3.

Approach: The approach for this Example will be to replace the Ni foil electrode of EMS with the electrodes synthesized in previous tasks and test them using an RDE setup by following the SOP formulated from Example 3. Based on these testing, the electrodes can be classified into three categories each operating in different E. coli concentration regions. Furthermore, the best electrode of the lot can be chosen and the testing conditions can be worked upon for this same probe to sense E. coli concentrations in all three levels.

Expected Results: At the end of this task, a working RDE probe with the best possible electrode configuration will be designed along with a SOP to operate it at set conditions under controlled environment for detection of E. coli in food items (lettuce, spinach).

The possibility of using a multi array probe with different catalyst materials (all three probes in one EMS) may be investigated.

### Example 5. Integration of the Optimum Electrode with Current Methodology for E. coli Detection in Food and Optimization of Detection Limit and Durability

A series of experiments will be run in Example 4 to choose the electrode composition which can be used for sensing E. coli in food in all specified concentration ranges. This electrode will be integrated with the EMS setup and a complete probe will be developed in this task.

Approach: In this task, the idea is to integrate the electrode composition and configuration selected as a result of extensive testing in Example 4 with the current EMS setup. A new EMS probe setup suitable for detecting E. coli concentration in food will thus be built. Further alterations in the process variables such as sample processing, same quantity, rotor speed will be varied to find the optimal operating conditions to achieve high sensitivity, durability and most importantly to achieve enhanced detection limit.

Expected Results: This Example should be able to deliver a complete generation 2 setup of EMS probe with the enhanced nanoparticle integrated electrode for sensing E. coli in the above-mentioned raw vegetables under controlled environment.

Further a multi array setup for the EMS may be designed based on the results from investigation of different electrode catalyst materials for different concentration ranges.

The embodiments of the present invention recited herein are intended to be merely exemplary and those skilled in the art will be able to make numerous variations and modifications to it without departing from the spirit of the present invention. Notwithstanding the above, certain variations and modifications, while producing less than optimal results, may still produce satisfactory results. All such variations and modifications are intended to be within the scope of the present invention as defined by the claims appended hereto.

What is claimed is:

1. An electrochemical sensor for detection of a pathogen, comprising:
a working electrode, a reference electrode, and a counter electrode;
wherein the working electrode includes a transition metal, and is contacted with a solution to be tested for the presence of a pathogen, the solution including an alkaline media for oxidation of the transition metal; and
wherein the sensor provides data to quantify the amount of any pathogen in the solution.

2. The electrochemical sensor of claim 1, wherein the transition metal is nickel.

3. The electrochemical sensor of claim 1, wherein the working electrode is a rotating disk electrode, and wherein the working electrode rotates in the solution including an alkaline media.

4. The electrochemical sensor of claim 2, wherein nickel hydroxide is oxidized to nickel oxyhydroxide at the surface of the working electrode.

5. The electrochemical sensor of claim 1, wherein the pathogen is chosen from Norovirus, Salmonella typhi, E. coli, Shigella, and Hepatitis A virus.

6. The electrochemical sensor of claim 5, wherein the pathogen is E. coli.

7. The electrochemical sensor of claim 1, wherein the alkaline media includes 0.01M KOH.

8. The electrochemical sensor of claim 1, wherein the sensor can quantify the amount of a pathogen in solution ranging from 10²-10¹⁰ CFU/mL.

9. The electrochemical sensor of claim 1, wherein the reference electrode includes platinum.

10. The electrochemical sensor of claim 1, wherein the counter electrode includes platinum.

11. An electrochemical sensor for detection of a pathogen, comprising:
a working electrode, a reference electrode, and a counter electrode;
wherein the working electrode includes a transition metal and graphene, and is contacted with a solution to be tested for the presence of a pathogen, the solution including an alkaline media for oxidation of the transition metal; and
wherein the sensor provides data to quantify the amount of any pathogen in the solution.

12. The electrochemical sensor of claim 1, wherein the transition metal is nickel.

13. The electrochemical sensor of claim 12, wherein the working electrode incudes graphene-layered nickel.

14. The electrochemical sensor of claim 11, wherein the working electrode is a rotating disk electrode, and wherein the working electrode rotates in the solution including an alkaline media.

15. The electrochemical sensor of claim 14, wherein nickel hydroxide is oxidized to nickel oxyhydroxide at the surface of the working electrode.

16. The electrochemical sensor of claim 11, wherein the pathogen is chosen from Norovirus, Salmonella typhi, E. coli, Shigella, and Hepatitis A virus.

17. The electrochemical sensor of claim 16, wherein the pathogen is E. coli.

18. The electrochemical sensor of claim 11, wherein the alkaline media includes 0.01M KOH.

19. The electrochemical sensor of claim 11, wherein the sensor can quantify the amount of a pathogen in solution ranging from 10²-10¹⁰ CFU/mL.
